(19) United States
(12) Patent Application Publication
(10) Pub. No.: US 2014/0277478 A1
(43) Pub. Date: Sep. 18, 2014

(54) SPINAL IMPLANT AND SURGICAL METHOD

(71) Applicant: K2M, INC., Leesburg, VA (US)

(72) Inventor: Jennifer Moore, Summit Point, WV (US)

(73) Assignee: K2M, INC., Leesburg, VA (US)

(21) Appl. No.: 13/798,959

(22) Filed: Mar. 13, 2013

(51) Int. Cl. A61F 2/44

(52) U.S. Cl. CPC A61F 2/442
USPC 623/17.16

(57) ABSTRACT

A spinal implant includes a substantially U-shaped body portion and a plate portion. The body portion has first, second, and third walls joined to one another such that the second and third walls define legs of the body portion. The body portion is configured and dimensioned to be positioned between first and second vertebral bodies. The plate portion has a main portion and a flange portion. The main portion has at least one first screw hole oriented towards the first vertebral body at an oblique angle relative to a horizontal axis of the body portion. The flange portion has a second screw hole oriented towards the first vertebral body with the second screw hole oriented substantially parallel to the horizontal axis of the body portion. The flange portion is coupled to the main portion such that the flange portion extends past the first surface of the main portion.

### BACKGROUND

1. Technical Field

The present disclosure relates to devices and methods for treating spinal conditions and, more particularly, to a spinal implant configured for positioning within the intervertebral space between adjacent vertebral bodies and methods of the same.

2. Background of Related Art

The human spine includes thirty-three vertebrae. The vertebrae interlock with one another to form a spinal column. Each vertebra has a cylindrical bony body (vertebral body), two pedicles extending from the vertebral body, a lamina extending from the pedicles, two wing-like projections extending from the pedicles, a spinous process extending from the lamina, a pars interarticularis, two superior facets extending from the pedicles, and two inferior facets extending from the lamina. The vertebrae are separated and cushioned by thin pads of tough, resilient fiber known as intervertebral discs. Intervertebral discs provide flexibility to the spine and act as shock absorbers during activity. A small opening (foramen) located between each vertebra allows passage of nerves. When the vertebrae are properly aligned, the nerves pass through without a problem. However, when the vertebrae are misaligned or a constriction is formed in the spinal canal, the nerves get compressed and may cause back pain, leg pain, or other neurological disorders.

### SUMMARY

In accordance with the present disclosure, a spinal implant is provided. The spinal implant includes a substantially U-shaped body portion and a plate portion. The body portion has first, second, and third walls. A first end of each of the second and third walls is joined to the first wall at opposing ends of the first wall such that the second and third walls define legs of the body portion. The body portion is configured and dimensioned to be positioned between first and second vertebral bodies. The plate portion has a main portion and a flange portion. The main portion has first and second surfaces and at least one first screw hole oriented towards the first vertebral body at an oblique angle relative to a horizontal axis of the body portion. The flange portion has a second screw hole oriented towards the first vertebral body with the second screw hole oriented substantially parallel to the horizontal axis of the body portion. The flange portion is coupled to the main portion such that the flange portion extends past the first surface of the main portion.

In embodiments, the spinal implant further includes a plurality of bone screws. Each bone screw is insertable through a corresponding screw hole of the plate and attachable to bone.

In embodiments, the plate portion further includes at least one locking mechanism, such that when a bone screw is inserted through a screw hole, the locking mechanism maintains the bone screw within the screw hole. The locking mechanism may include a locking ring positionable in the screw hole for retaining the bone screw and/or may include a lip formed in each screw hole that is deformable by the screw to lock the screw to the lip.

In embodiments, the main portion of the plate portion has a pair of first screw holes and a cut-out defined about each of the first screw holes on each end thereof. More specifically, the cut-outs may be oriented towards the first vertebral body at an oblique angle relative to the horizontal axis of the body portion.

In embodiments, the substantially U-shaped body portion and the plate portion are unitarily formed with one another.

In embodiments, the body portion is configured and dimensioned so that when the body portion is inserted between the first and second vertebral bodies the first surface of the body walls contacts the first vertebral body and the second surface of the walls contacts the second vertebral body. Further, the first and second surfaces of the body portion may be substantially coplanar with first and second surfaces of the main portion of the plate portion.

In embodiments, the spinal implant further includes at least one first bone screw insertable through the at least one first screw hole into the first vertebral body at an oblique angle relative to the horizontal axis of the body portion and a second bone screw insertable through the second screw hole into the first vertebral body in substantially parallel orientation relative to the horizontal axis of the body portion.

Also provided in accordance with the present disclosure is a method of performing surgery. The method includes providing a surgical implant similar to any of the embodiments above, preparing an intervertebral space between first and second vertebral bodies, inserting the body portion of the surgical implant into the prepared intervertebral space, inserting at least one first screw through the at least one first screw hole of the main portion of the plate portion and into the first vertebral body, and inserting a second screw through the second screw hole of the flange portion of the plate portion and into the first vertebral body.

In embodiments, the body portion is a substantially U-shaped body portion and the method further includes packing the body portion with material.

In embodiments, inserting the at least one first screw and/or inserting the second screw includes locking the screw within the plate portion using a screw locking mechanism.

In embodiments, the at least one first screw is inserted through the at least one screw hole of the main portion of the plate portion at an oblique angle relative to the horizontal axis of the body portion and into the first vertebral body.

In embodiments, the second screw is inserted through the second screw hole of the flange portion of the plate portion in substantially parallel orientation relative to the horizontal axis of the body portion and into the first vertebral body.

### BRIEF DESCRIPTION OF THE DRAWINGS

Various aspects and features of the present disclosure are described with reference to the accompanying drawing figures, wherein:

FIG. 1A is a bottom, front, perspective view of one embodiment of a spinal implant provided in accordance with the present disclosure;

FIG. 1B is a top, front, perspective view of the spinal implant of FIG. 1A;

FIG. 1C is a side view of the spinal implant of FIG. 1A;

FIG. 2A is a side view of a bone screw configured for use with the spinal implant of FIG. 1A;

FIG. 2B is a side, cross-sectional view of the bone screw of FIG. 2A;

FIG. 2C is a top view of the bone screw of FIG. 2A; and

FIG. 3 is a top, perspective view of a locking ring configured for use with the spinal implant of FIG. 1A.

### DETAILED DESCRIPTION OF THE EMBODIMENTS

Embodiments of the present disclosure are now described in detail with reference to the drawings, wherein like reference numerals identify similar or identical elements. In the drawings and in the description that follows, the term “proximal” will refer to the end of device that is closest to the operator, while the term “distal” will refer to the end of the device that is farthest from the operator. In addition, the term “cephalad” is used in this application to indicate a direction toward a patient's head, whereas the term “caudad” indicates a direction toward the patient's feet. Further still, for the purposes of this application, the term “medial” indicates a direction toward the middle of the body of the patient, whilst the term “lateral” indicates a direction toward a side of the body of the patient (i.e., away from the middle of the body of the patient). The term “posterior” indicates a direction toward the patient's back, and the term “anterior” indicates a direction toward the patient's front.

Referring to FIGS. 1A-1C, one embodiment of a spinal implant provided in accordance with the present disclosure is shown generally designated by reference numeral 300. Spinal implant 300 is a unitary implant including a plate portion 310 and a body portion 340. Body portion 340 defines a generally U-shaped configuration, while plate portion 310 includes a main portion 312 and a flange portion 314. Each of these portions of spinal implant 300 will be described in detail below. Spinal implant 300 is configured for use with a plurality of bone screws 170 (FIGS. 2A-2C) for securing spinal implant 300 between adjacent vertebral bodies, as will also be described in detail below.

The various portions of spinal implant 300 and bone screws 170 (FIGS. 2A-2C) configured for use therewith may be formed from various similar or different materials, depending on a particular purpose. For example, plate portion 310 and body portion 340 may be formed from the same or different materials. In particular, body portion 340 may be formed from a metallic material (similar or different to those of plate portion 310) or a non-metallic material, e.g., polymeric materials such as polyetheretherketone (PEEK) or organic materials such as bone, while plate portion 310 may be formed from a metallic material, e.g., titanium, titanium alloy, or cobalt chrome (CoCr) or a non-metallic synthetic material, e.g., polymeric materials such as PEEK, nylon absorbable polymers such as polyglycolides, polylactides, polycaprolactone, etc. Bone screws 170 (FIGS. 2A-2C) may be formed from titanium, titanium alloy, CoCr or other suitable metal or polymeric material compatible with plate portion 310. With additional reference to FIGS. 2A-2C, in some embodiments, bone screw 170, or at least head 174 thereof, is formed from a harder material, e.g., titanium alloy or CoCr, than the material, e.g., pure titanium, used to form plate portion 310, the importance of which will be described in greater detail below.

Plate portion 310 of spinal implant 300, as mentioned above, includes a main portion 312 and a flange portion 314 extending from main portion 312. Flange portion 314 is coupled to a first edge 313 of main portion 312 via an extension portion 316 such that flange portion 314 extends vertically and outwardly from first edge 313 of main portion 312. Despite flange portion 314 being offset vertically and outwardly from main portion 312, first faces 317, 318 of main portion 312 and flange portion 314, respectively, define substantially planar surfaces disposed in substantially parallel orientation relative to one another. Main portion 312 and flange portion 314 are monolithically formed, e.g., such that plate portion 310 of spinal implant 300 defines a unitary or monolithic configuration, although main portion 312 and flange portion 314 may alternatively be secured to one another in any suitable fashion.

Main portion 312 of plate portion 310 of spinal implant 300 includes a first face 317, a second, opposite face 319, a first surface 321, and a second, opposite surface 322. First and second surfaces 321, 322, respectively, of main portion 312 of plate portion 310 of spinal implant 300 are configured for contacting the opposed surfaces of the adjacent vertebral bodies between which spinal implant 300 is positioned. First and second surfaces 321, 322 may define a plurality of ridges (not shown) configured to frictionally engage the opposed surfaces of the adjacent vertebral bodies to maintain plate portion 310 in position relative to the adjacent vertebral bodies and to inhibit spinal implant 300 from backing out of the intervertebral space.

Main portion 312 of plate portion 310 of spinal implant 300 further defines a plurality of screw holes 324 extending through main portion 312. In particular, main portion 312 of plate portion 310 may define first and second screw holes 324 positioned towards the respective first and second sides thereof. Each screw hole 324 is obliquely angled relative to first face 317 of main portion 312 of plate portion 310 and a horizontal axis “X-X” of body 340 (see FIG. 1C), i.e., screw holes 324 extend in non-perpendicular orientation relative to first face 317 of main portion 312 of plate portion 310 and the horizontal axis “X-X” of body 340 (see FIG. 1C). Specifically, screw holes 324 extend from a first end adjacent to first face 317 and second surface 322, to a second end adjacent to second face 319 and first surface 321. This angled configuration of screw holes 324 directs bone screws 170 (FIGS. 2A-2C) inserted therethrough at similar oblique angles towards one of the vertebral bodies for engagement of bone screws 170 (FIGS. 2A-C) within the vertebral body despite main portion 312 being vertically displaced, e.g., vertically offset, relative to the vertebral body into which the bone screws 170 (FIGS. 2A-2C) extending through screw holes 324 is to engage.

Main portion 312 of plate portion 310 of spinal implant 300 also includes a plurality of cut-outs 332, 333 defined therein and positioned to surround screw holes 324. More specifically, first face 317 of main portion 312 defines a substantially planar configuration except for a portion of each of a pair of cut-outs 332, which are disposed about screw holes 324. The remaining portions of cut-outs 332 are defined within second surface 322. Second surface 322 is otherwise substantially planar in configuration. Second face 319 also defines a substantially planar configuration except for a portion of each of a pair of cut-outs 333, which are disposed about screw holes 324. The remaining portions of cut-outs 333 are defined within first surface 321. Thus, cut-outs 332, 333, like screw holes 324, are obliquely defined relative to main portion 312 of plate portion 310, thus directing bone screws 170 (FIGS. 2A-2C) through main portion 312 of plate portion 310 of spinal implant 300 in oblique orientation relative to spinal implant 300.

Main portion 312 of plate portion 310 further defines a lip 335 defined within each cut-out 332, 333, and positioned about and extending into each screw hole 324. Lips 335 are configured to abut heads 174 of bone screws 170 to inhibit heads 174 of bone screws 170 from passing through screw holes 324. Lips 335 will be described in greater detail below. Further, as will also be described in greater detail below, a locking mechanism 190 (FIG. 3) may be positionable within or incorporated into cut-out 332, 333 adjacent screw holes 324 for securing heads 174 of bone screws 170 therein (see FIGS. 2A-2C).

With continued reference to FIGS. 1A-1C, flange portion 314 of plate portion 310 of spinal implant 300, as mentioned above, is coupled to first edge 313 of main portion 312 via an extension portion 316. Flange portion 314 defines a first face 318 and a second, opposite face 336. Flange portion 314 further defines one or more screw holes 338 extending therethrough from first face 318 to second face 336 of flange portion 314. Screw hole 338 extends in substantially perpendicular orientation relative to first face 318 of flange portion 314 of plate portion 310 and in substantially parallel orientation relative to the horizontal axis “X-X” of body portion 340 (see FIG. 1C).

The above-described configuration, wherein flange portion 314 is disposed above main portion 312 of plate portion 310 allows flange portion 314 to be positioned adjacent, e.g., vertically aligned with, and in contact with a surface of one of the vertebral bodies. Thus, with flange portion 314 position adjacent (vertically aligned with) one of the vertebral bodies, and with screw hole 338 extending perpendicularly through flange portion 314, screw hole 338 is positioned to guide bone screws 170 (FIGS. 2A-2C) into the vertebral body for securement therein in substantial parallel orientation relative to the horizontal axis “X-X” of body portion 340. With spinal implant 300 so implanted, flange portion 314 abuts and is secured to the face of a vertebral body, and main portion 312 is offset from flange portion 314 by extension portion 316 and is disposed substantially between the vertebral bodies adjacent body portion 340 with the surfaces of the body and main portions 340, 312, respectively, substantially co-planar with one another. Similar to screw holes 324 of main portion 312 of plate portion 310, screw hole 338 of flange portion 314 further defines a lip 339 disposed about and extending into screw hole 338. Lip 339 is configured to abut head 174 of bone screw 170 to inhibit head 174 of bone screw 170 from passing through screw hole 338 (see FIGS. 2A-2C). Further, as will be described in greater detail below, a locking mechanism 190 (FIG. 3) may be positionable within or incorporated into screw hole 338 for securing head 174 of bone screw 170 therein (see FIGS. 2A-2C).

Continuing with reference to FIGS. 1A-1C, generally U-shaped body portion 340 of spinal implant 300 is formed from a first wall 342 and second and third walls 344, 346 which extend from first wall 342 to define the legs of the generally U-shaped body 340. Body 340 defines a horizontal axis “X-X,” and is configured such that walls 344, 346 extend in substantially parallel orientation relative to horizontal axis “X-X.” Second and third walls 344, 346 are coupled, e.g., monolithically formed, joined, or otherwise engaged, with plate portion 310 of spinal implant 300 such that screw holes 324 of main portion 312 of plate portion 310 are disposed at an oblique angled relative to axis “X-X,” while screw hole 338 of flange portion 314 of plate portion 310 is substantially parallel to axis “X-X.” Body portion 340 and main portion 312 of plate portion 310 may be oriented such that first and second surfaces 347, 348, respectively, of body portion 340 and first and second surfaces 321, 322, respectively, of main portion 312 of plate portion 310, are substantially coplanar with one another. Thus, upon insertion into the intervertebral space, spinal implant 300 is positionable such that first surfaces 347, 321 are configured to contact a first vertebral body, while second surfaces 348, 322 are configured to contact a second vertebral body. First and second surfaces 347, 348 may define a plurality of ridges 349 arranged in rows and configured to frictionally engage the opposed surfaces of the adjacent vertebral bodies to maintain body portion 340 in position relative to the adjacent vertebral bodies and to inhibit spinal implant 300 from backing out of the intervertebral space.

Body portion 340 may further include a pair of engagement recesses 354 defined within the outer surfaces of walls 344, 346 and configured to receive a portion of an insertion tool (not shown) for coupling body 340 to the insertion tool (not shown) to facilitate insertion and placement of spinal implant 300 within the intervertebral space. Additionally or alternatively, plate portion 310 may include an engagement recess or other suitable feature (not shown) configured to engage the insertion tool (not shown) for similar purposes.

Referring now to FIGS. 2A-2C, in conjunction with FIGS. 1A-1C, a bone screw 170 configured for use with spinal implant 300 is shown. As can be appreciated, a plurality of bone screws 170 are configured to secure spinal implant 300 between adjacent vertebral bodies. However, as bone screws 170 are similar to one another, only one is described in detail herein. It is also contemplated that other suitable bone screws 170 be provided for use with spinal implant 300.

Bone screw 170 generally includes a shank 172 and a head 174. Shank 172 defines a distal tip 176 and pitched threading 178 disposed about shank 172. Distal tip 176 and pitched threading 178 facilitate driving bone screw 170 into bone and securement of bone screw 170 therein. Head 174 of bone screw 170 defines a tool-engaging recess 180 and an annular flange 182 defining a greater diameter than shank 172. Tool-engaging recess 180 defines a configuration, e.g., a 6-point star or other suitable configuration, shaped complementary to a driving end of an insertion tool (not shown) to facilitate engagement of the insertion tool (not shown) with head 174 of bone screw 170 for rotational driving of bone screw 170 into a vertebral body. Flanges 182 of heads 174 of bone screws 170 are configured to be seated within lips 335, 339 of screw holes 324, 338, respectively, to inhibit bone screws 170 from passing through screw holes 324, 338. As mentioned above, in some embodiments, bone screws 170 (or heads 174 thereof) are formed from a harder material than plate portion 310 of spinal implant 300 with threads (not shown) provided on heads 174 to engage and deform lips 335, 339 in screw holes 324, 338 to lock bone screws 170 within plate portion 310 of spinal implant 300. See, for example, U.S. Pat. No. 6,322,562 to Wolter, the entire contents of which are hereby incorporated by reference herein. Alternatively, other suitable locking mechanisms 190, e.g., locking rings 192, may be provided.

Turning now to FIG. 3, in conjunction with FIGS. 1A-2C, as mentioned above, locking mechanism 190 may include a locking ring 192 configured for engagement within one or more of screw holes 324, 338 to retain screw head 174 between locking ring 192 and the lip 335, 339 of the screw hole 324, 338, respectively. Locking ring 192 defines a generally annular configuration having a disconnect, or interruption 194. Thus, with interruption 194 formed in locking ring 192, locking ring 192 defines first and second resilient legs 195, 197. In an at-rest condition, locking ring 192 defines a diameter that approximates or is larger than a diameter of screw holes 324, 338. In a compressed condition, legs 195, 197 are urged inwardly to define a diameter of locking ring 192 that is smaller than the diameter of screw holes 324, 338, thus permitting locking ring 192 to be inserted into the screw hole 324, 338 after insertion of bone screw 170 therethrough to retain head 174 of bone screw 170 between lip 335, 339 and locking ring 192. The resilient bias of locking ring 192 towards the at-rest condition retains locking ring 192 in position, thus inhibiting bone screw 170 from backing out of the screw hole 324, 338. Further, an annular slot (not explicitly shown) may be formed within each screw hole 324, 338 for receiving a locking ring 192 upon return of locking ring 192 under bias towards the at-rest position, thus securing locking ring 192 within the screw holes 324, 338. In some embodiments, plate portion 310 of spinal implant 300 may be pre-loaded with locking rings 192. In such embodiments, upon insertion of bone screws 170 into screw holes 324, 338, heads 174 of bone screws 170 urge locking ring 192 to expand beyond the at-rest position, thus permitting passage of heads 174 through locking rings 192. Upon return of locking rings 192 towards the at-rest position, locking rings 192 retains heads 174 within screw holes 324, 338, similarly as described above. Spinal implant 300 need not be pre-loaded with locking rings 192. Further, other suitable locking mechanisms (not shown) for securing bone screws 170 within plate portion 310 of spinal implant 300 are also contemplated.

With reference to FIGS. 1A-3, the insertion of spinal implant 300 into the intervertebral space between adjacent vertebral bodies during the course of a spinal surgical procedure is described. Initially, the intervertebral space is prepared, e.g., damaged or diseased tissue is removed. Next, spinal implant 300, lead by first wall 342 of body portion 340, is inserted into the intervertebral space between the adjacent vertebral bodies. At this point, or prior thereto, if the particular procedure so dictates, the interior space defined by the generally U-shaped body 340 may be packed with bone in-growth material, drugs, or other suitable materials or compounds.

Once spinal implant 300 is moved into proper position, first and second bone screws 170 are inserted through the respective first and second screw holes 324 of main portion 312 of plate portion 310 and are driven into one of the adjacent vertebral bodies. More specifically, due to the obliquely angled configuration of screw holes 324 relative to axis “X-X,” as mentioned above, bone screws 170 are guided through screw holes 324 and into the vertebral body. Next, a third bone screw 170 is inserted through screw hole 338 of flange portion 314 of plate portion 310 and are likewise driven into the vertebral body, e.g., the same vertebral body within which the bone screws 170 extending through screw holes 324 are secured. However, it is also contemplated that the bone screw 170 extending through screw hole 338 be inserted first, followed by the bone screws 170 inserted through screw holes 324, as the procedure may dictate. The parallel orientation of screw hole 338 guides bone screws 170 therethrough and into the vertebral body. Once bone screws 170 are positioned as desired, locking rings 192 may be inserted into screw holes 324, 338 to lock the heads 174 of bone screws 170 therein, thus retaining spinal implant 300.

It will be understood that various modifications may be made to the embodiments of the present disclosure. Therefore, the above description should not be construed as limiting, but merely as exemplifications of embodiments. Those skilled in the art will envision other modifications within the scope and spirit of the present disclosure.

What is claimed is:

1. A spinal implant comprising:
a substantially U-shaped body portion having first, second, and third walls, a first end of each of the second and third walls joined to the first wall at opposing ends of the first wall such that the second and third walls define legs of the substantially U-shaped body portion, the substantially U-shaped body portion configured and dimensioned to be positioned between first and second vertebral bodies;
a plate portion having a main portion and a flange portion, the main portion having first and second surfaces and at least one first screw hole oriented towards the first vertebral body at an oblique angle relative to a horizontal axis of the substantially U-shaped body portion, the flange portion having a second screw hole oriented towards the first vertebral body with the second screw hole oriented substantially parallel to the horizontal axis of the substantially U-shaped body portion, the flange portion coupled to the main portion such that the flange portion extends past the first surface of the main portion.

2. The spinal implant according to claim 1, further comprising a plurality of bone screws, each bone screw insertable through a corresponding screw hole of the plate and attachable to bone.

3. The spinal implant according to claim 1, wherein the plate portion further comprises at least one locking mechanism, such that when a bone screw is inserted through a screw hole, the locking mechanism maintains the bone screw within the screw hole.

4. The spinal implant according to claim 3, wherein the locking mechanism comprises a locking ring positionable in the screw hole for retaining the bone screw.

5. The spinal implant according to claim 3, wherein the locking mechanism comprises a lip formed in each screw hole, with the plate portion made of a first material and the screws made of a second, harder material such that the screw locks to the lip upon engagement of the screw with the lip.

6. The spinal implant according to claim 1, wherein the main portion of the plate portion has a pair of first screw holes and a cut-out defined about each of the first screw holes on each end thereof.

7. The spinal implant according to claim 6, wherein the cut-outs oriented towards the first vertebral body at an oblique angle relative to the horizontal axis of the substantially U-shaped body portion.

8. The spinal implant according to claim 1, wherein the substantially U-shaped body portion and the plate portion are unitarily formed with one another.

9. The spinal implant according to claim 1, wherein the substantially U-shaped body portion is configured and dimensioned so that when the substantially U-shaped body portion is inserted between the first and second vertebral bodies the first surface of the body walls contacts the first vertebral body and the second surface of the walls contacts the second vertebral body.

10. The spinal implant according to claim 1, wherein the first and second surfaces of the substantially U-shaped body portion are substantially coplanar with first and second surfaces of the main portion of the plate portion.

11. The spinal implant according to claim 1, further comprising at least one first bone screw insertable through the at least one first screw hole into the first vertebral body at an oblique angle relative to the horizontal axis of the substantially U-shaped body portion, and a second bone screw insertable through the second screw hole into the first vertebral body in substantially parallel orientation relative to the horizontal axis of the substantially U-shaped body portion.

12. A method of performing surgery, comprising:
providing a surgical implant, including:
a body portion; and
a plate portion having a main portion and a flange portion, the main portion having at least one first screw hole oriented at an oblique angle relative to a horizontal axis of the body portion, the flange portion having a second screw hole oriented substantially parallel to the horizontal axis of the substantially U-shaped body portion;
preparing an intervertebral space between first and second vertebral bodies to receive the body portion of the surgical implant;
inserting the body portion of the surgical implant into the prepared intervertebral space;
inserting at least one first screw through the at least one first screw hole of the main portion of the plate portion and into the first vertebral body; and
inserting a second screw through the second screw hole of the flange portion of the plate portion and into the first vertebral body.

13. The method according to claim 12, wherein the body portion is a substantially U-shaped body portion and wherein the step of inserting the body portion further includes packing the body portion with material.

14. The method according to claim 12, wherein at least one of the steps of inserting the at least one first screw and inserting the second screw includes locking the screw within the plate portion using a screw locking mechanism.

15. The method according to claim 12, wherein the at least one first screw is inserted through the at least one screw hole of the main portion of the plate portion at an oblique angle relative to the horizontal axis of the body portion and into the first vertebral body.

16. The method according to claim 12, wherein the second screw is inserted through the second screw hole of the flange portion of the plate portion in substantially parallel orientation relative to the horizontal axis of the body portion and into the first vertebral body.
